(19) United States
(12) Patent Application Publication
(10) Pub. No.: US 2020/0261024 A1
(43) Pub. Date: Aug. 20, 2020

(54) OESOPHAGEAL ELECTRODE PROBE AND DEVICE FOR CARDIOLOGICAL TREATMENT AND/OR DIAGNOSIS

(71) Applicant: HOCHSCHULE OFFENBURG, Oftenburg (DE)

(72) Inventors: Mathias HEINKE, Rudolstadt (DE); Marco SCHALK, Schifferstadt (DE)

(21) Appl. No.: 16/761,930

(22) PCT Filed: Nov. 7, 2018
(86) PCT No.: PCT/EP2018/080382

§ 371 Date: May 6, 2020

(30) Foreign Application Priority Data
Nov. 7, 2017 (DE) 10 2017 010 318.6

(51) Int. Cl. A61B 5/00; A61B 5/053; A61N 1/05; A61B 5/042; A61B 5/0205; A61B 5/145; A61B 18/14; A61N 1/36; A61N 1/365

(52) U.S. Cl. CPC A61B 5/687; A61B 5/0538; A61N 1/0517; A61B 5/6853; A61B 5/0421; A61B 5/02055; A61B 2018/00577; A61B 18/1492; A61N 1/36071; A61N 1/36521; A61N 1/3614; A61B 5/0422; A61B 5/14539

(57) ABSTRACT

An oesophageal electrode probe for bioimpedance measurement and/or for neurostimulation is provided; a device for transoesophageal cardiological treatment and/or cardiological diagnosis is also provided; a method for the open-loop or closed-loop control of a cardiological catheter ablation device and/or a cardiological, circulatory and/or respiratory support device is also provided. The oesophageal electrode probe comprises a bioimpedance measuring device for measuring the bioimpedance of at least one part of tissue surrounding the oesophageal electrode probe. The bioimpedance device comprises at least one first and one second electrode. The at least one first electrode is arranged on a side of the oesophageal electrode probe facing towards the heart. The at least one second electrode is arranged on a side of the oesophageal electrode probe facing away from the heart. The device comprises the oesophageal electrode probe and a control and/or evaluation device.

### CROSS-REFERENCE TO RELATED APPLICATIONS

This application is a national phase entry under 35 U.S.C. 371 of International Application No. PCT/EP2018/080382, filed Nov. 7, 2018, which claims priority to DE Application No. 10 2017 010 318.6, filed Nov. 7, 2017, the entire contents of which are incorporated by reference herein.

### SUMMARY

The disclosure relates to an esophageal electrode probe or an esophageal catheter for bioimpedance measurement and/or for neurostimulation and to a device for transesophageal cardiological treatment and/or cardiological diagnosis, which includes an esophageal electrode probe. Furthermore, the disclosure relates to a method for controlling or regulating an ablation device for performing a cardiac ablation (in particular a cardiac catheter ablation) and/or a cardiac, circulatory and/or respiratory support device for cardiosynchronous cardiac, circulatory and/or respiratory support.

In particular, the disclosure relates to a device and an esophageal electrode probe for directed transesophageal cardiological stimulation, electrocardiography, cardioversion, bioimpedance measurement and/or neurostimulation for transesophageal electrophysiological examinations of the heart for the diagnosis and therapy of arrhythmias, for neurostimulation by transesophageal stimulation of the ascending neural pathways of the spinal cord for suppression of neural conduction for supprection of pain perception in the brain and for neurological electrophysiological examinations, for measurements of the cardiac output, the bioimpedances in different measurement sections of the heart and the esophagus, as well as other hemodynamic, electrocardiographic and impedance-cardiographic parameters in the context of the temporary transesophageal atrial and/or ventricular stimulation, atrial and/or ventricular electrocardiography, bioimpedance measurement and for optimization of the energy output of heat and cold energy as well as laser energy in the context of catheter ablation for tachycardia arrhythmias to avoid esophageal injuries. The disclosure further relates to an esophageal electrode probe and a cardiac, circulatory and/or respiratory support device for physiological cardiosynchronous cardiac, circulatory and/or respiratory support, e.g. for the treatment of high-risk patients in interventional cardiology and for the treatment of cardiogenic shock.

Various methods and esophageal electrode probes for transesophageal stimulation and electrocardiography of the heart for diagnosis and therapy of arrhythmias and transesophageal impedance cardiography for measurement of the cardiac output are known in the prior art (DD 247608A1, DD 210207A1, DD 149610A1, DD 133400A1, DE 102004001626A1, DE 3327561A1, US 2003/0097167A1, US 2002/0198583A1, U.S. Pat. No. 4,836,214, WO 81/03428, U.S. Pat. Nos. 4,574,807, 5,056,531, 5,967,977, 6,006,138, 5,431,696, 4,304,239, 5,191,885, 5,571,150, 5,370,679, 5,431,696, D. J. McEneaney: An Esothoracic Electrode for Electrophysiological Studies: Journal of Electrophysiology Vol. 35 Supplement 2002, 151-157, H. R. Andersen, et al.: TransEsophageal Pacing, PACE 6, 1983. 674-679). Methods and devices for ablation for the treatment of arrhythmias, such as atrial fibrillation, are also known from the prior art. For the treatment of atrial fibrillation by isolation of the respiratory veins in the left atrium, for example, high-frequency, cryo-, ultrasound or laser ablation can be used.

Ablation or catheter ablation can, however, result in life-threatening injuries to the esophagus. To reduce or avoid this, catheter ablation is monitored in some clinics using esophageal temperature measurement. An exemplary esophageal probe for temperature monitoring is the esophageal probe from the company BISPING Medizintechnik GmbH, which enables temperature monitoring from −20° to +65° and covers the entire left atrium by 12 temperature sensors. However, continuous temperature monitoring is often inadequate to ensure a high level of safety for ablation or catheter ablation, since tissue injuries are already present when a relevant rise in temperature is detected.

Cardiological treatments such as transesophageal left-atrial and/or left-ventricular stimulation as part of a temporary cardiac resynchronization therapy, sometimes cause considerable pain in the treated patients.

It is therefore an object of the disclosure to provide an improved esophageal electrode probe and a device which enable more patient-friendly and/or less painful cardiological treatments and/or measurements.

This object is achieved by an esophageal electrode probe and a device for transesophageal cardiological treatment and/or diagnosis according to the independent claims. Preferred embodiments are subject of the dependent claims.

It has surprisingly been found that a considerable reduction in the tissue damage caused by cardiac ablation or catheter ablation can be achieved by monitoring the bioimpedance with the aid of a novel esophageal electrode probe, in which electrodes for measuring the bioimpedance are arranged both on the side of the esophageal electrode probe facing the heart and on the side facing away from the heart. With the help of the proposed esophageal electrode probe, it is possible to monitor tissue changes by means of a transesophageal bioimpedance measurement with the electrodes facing the heart and to compare them with a bioimpedance measurement with the electrodes facing away from the heart in order to derive and/or present information on the basis of which the further progress of the ablation or catheter ablation can be changed. At the same time, transesophageal hemodynamic monitoring can be performed.

It has also surprisingly been found that transesophageal neurosimulation on the side of an esophageal electrode probe facing away from the heart makes it possible to achieve a pain reduction in transesophageal cardiac treatment, such as in transesophageal cardiac stimulation or transesophageal cardioversion to terminate atrial fibrillation.

A first aspect of the disclosure relates to an improved esophageal electrode probe and an improved device for transesophageal bioimpedance monitoring, e.g. in ablation or catheter ablation and/or cardiac, circulatory and/or respiratory support. The ablation or catheter ablation can e.g. be a high-frequency, cryogenic, ultrasound or laser ablation, e.g. an ablation to treat atrial fibrillation by isolating the respiratory veins in the left atrium.

A second aspect of the disclosure (alternatively or in addition to the first aspect) relates to an improved esophageal electrode probe and a device for transesophageal cardiac stimulation, in particular for transesophageal left-atrial and/or left-ventricular stimulation in the context of diagnosis and therapy of bradycardic and tachycardic arrhythmias, such as initiation and termination of AV node reentry tachycardia (AVNRT), AV reentry tachycardia (AVRT) and atrial flutter, as well as for transesophageal left-ventricular stimulation as part of a temporary cardiac resynchronization therapy or an antibradycardic temporary cardiac stimulation. The esophageal electrode probe has at least one electrode on the side of the esophageal electrode probe facing away from the heart for neurostimulation. The proposed transesophageal neurostimulation enables pain reduction and a reduction in the sensation threshold of the transesophageal cardiological stimulation.

An exemplary esophageal electrode probe according to a first aspect of the disclosure includes a bioimpedance measuring device for measuring the bioimpedance of at least part of the tissue surrounding the probe. The bioimpedance measuring device includes at least one first electrode and at least one second electrode. The at least one first electrode is arranged on a first side of the probe. The at least one second electrode is arranged on a second side of the probe. The first side and the second side of the probe are opposite in the radial direction of the probe. When the esophageal electrode probe is inserted into the patient's esophagus, the first side of the probe faces the heart and the second side of the probe faces away from the heart. The esophageal electrode probe can furthermore have a marking, which makes it possible to identify the side facing the heart and the side facing away from the heart.

The esophageal electrode probe may further comprise a neurostimulation device. The neurostimulation can optionally take place as cardiac neurostimulation in the direction of the heart and/or non-cardiac neurostimulation in the direction of the spine. The neurostimulation device can comprise e.g. at least one electrode for transesophageal neurostimulation of the at least part of the tissue surrounding the probe by means of electric pulses with a frequency of 100 bpm to 3000 bpm, preferably between 1500 bpm and 2000 bpm, a strength of about 5V to 100V, and a duration of 3 seconds to 10 minutes, preferably between 3 and 30 seconds. Alternative and further parameters (not for temporary transesophageal stimulation) of invasive neurostimulation are described in the Journal of Cardiovasc. Electrophysiol., Vol. 21, pp. 193-199, February 2010. The at least one electrode for neurostimulation can be arranged on the second side of the esophageal electrode probe facing away from the heart. The at least one electrode for neurostimulation is in particular adapted to achieve neurostimulation for pain reduction in the case of transesophageal electrostimulation of at least part of the tissue surrounding the esophageal electrode probe. It is possible for the esophageal electrode probe to only have a neurostimulation device, but not a bioimpedance measuring device.

The esophageal electrode probe can basically be constructed like a conventional esophageal electrode probe. For example, the esophageal electrode probe can have an elongated, substantially cylindrical probe body. For example, the probe body can be made of a flexible material. The probe body has a proximal and a distal end, the axis of the probe body substantially coinciding with the insertion direction of the esophageal electrode probe into the patient's esophagus. In contrast to conventional esophageal electrode probes, the present disclosure proposes that electrodes for bioimpedance measurement be arranged both on the side of the probe facing the heart and on the side facing away from the heart. As an alternative or in addition, it is proposed that electrodes for neurostimulation be arranged on the side of the probe facing away from the heart.

The electrodes can be attached to the probe body. The esophageal electrode probe preferably has an inflatable catheter balloon made of a suitable biocompatible elastic material, the catheter balloon being attached to the probe body by means of a more suitable fastening device. The electrodes of the bioimpedance measuring device and/or the neurostimulation device can be arranged on the catheter balloon. When the catheter balloon is inflated, the electrodes preferably come into contact with the patient's esophagus.

The individual electrodes can be conventional electrodes for impedance measurement and/or neurostimulation. They can have a substantially semi-cylindrical or semi-spherical shape, the curved surface coming into contact with the tissue to be examined. The electrodes can be made of a biocompatible conductive material, such as metal or conductive plastic/rubber.

The electrodes can furthermore be arranged in groups, each including one or more rows of electrodes, wherein there can be a constant or different distance between the individual electrodes. The electrodes can be arranged in arbitrary matrix form with a variable electrode-myocardium distance. For example, the electrodes can be arranged in rows in the longitudinal direction of the esophageal electrode probe.

The esophageal electrode probe can further comprise further devices for the treatment and/or examination of the heart and/or other body organs in the vicinity of the esophagus. In particular, the esophageal electrode probe can further comprise:
a stimulation device with at least one electrode for transesophageal cardiac stimulation, the electrode being arranged on the side of the esophageal electrode probe facing the heart; and or
an electrography device with at least one electrode or sensor for electrography measurement; and or
an echocardiography device with at least one ultrasound sensor for echocardiography measurement; and or
a temperature measuring device with at least one temperature sensor for measuring the temperature of at least part of the tissue surrounding the esophageal electrode probe; and or
a pH value measuring device with at least one pH value sensor for pH value measurement of at least part of the tissue surrounding the esophageal electrode probe.

This makes it possible to carry out several different examinations and/or treatments simultaneously or in a timely manner with one probe, such as electrocardiography, echocardiography, and cardiac stimulation. The measurement data obtained from the individual devices and/or sensors can be combined or evaluated together, for example to improve the precision of a cardiac treatment (such as cardiac ablation or cardiac catheter ablation or cardiac stimulation) and/or cardiac examination. For example, temperature data and/or echography data and/or electrocardiography data can be combined with the impedance signals or impedance measurement data in order to improve the precision of a cardiac ablation or cardiac catheter ablation.

Furthermore, a device for transesophageal cardiological treatment and/or diagnosis is proposed, which includes an esophageal electrode probe according to the disclosure. The device for transesophageal cardiological treatment and/or diagnosis further includes a control and/or evaluation device, the control and/or evaluation device being in signal connection with the esophageal electrode probe. The control and/or evaluation device is adapted to receive and evaluate signals from the at least part of the electrodes of the esophageal electrode probe and/or to send signals to at least part of the electrodes (such as the electrodes of the neurostimulation device).

The control and/or evaluation device can in particular be in signal connection with the bioimpedance measuring device of the esophageal electrode probe and can be adapted to compare the signals received by the electrodes of the bioimpedance measuring device and to generate a check signal based on the comparison of the received signals.

Alternatively or in addition, the control and/or evaluation device can be in signal connection with an electrography device of the esophageal electrode probe and can be adapted to compare the signals received by the electrodes of the electrography device and to generate a check signal based on the comparison of the received signals.

It is also possible to generate the check signal on the basis of both the received signals of the bioimpedance measuring device and the received signals of the electrography device.

The check signal can be a status signal that indicates the status and/or the value of at least one treatment-relevant parameter. The treatment can be, for example, a cardiac ablation or cardiac catheter ablation, and the check signal can indicate the status or the value of at least one parameter of the cardiac ablation or cardiac catheter ablation, e.g. energy output, temperature, progress of cardiac ablation or cardiac catheter ablation, tissue damage occurring, etc. The status signal can also be the status and/or the value of at least one treatment-relevant parameter of a cardiosynchronous cardiac, circulatory and/or respiratory support, such as start, end, scope, etc. The status signal can also be an optical, acoustic or haptic warning signal, which indicates that at least one treatment-relevant parameter is outside a permissible value range or is larger/smaller than a predetermined threshold value.

The check signal can also be a control signal for controlling or regulating a device for treating a patient, such as a cardiac ablation device or cardiac catheter ablation device and/or a cardiac, circulatory and/or respiratory support device for physiological cardiosynchronous cardiac, circulatory and/or respiratory support. The control signal can control or regulate at least one parameter of the ablation or catheter ablation and/or the cardiac, circulatory and/or respiratory support, such as intensity, temperature, duration or spatial extent of the ablation; volume, flow rate, oxygen and/or carbon dioxide transfer, temperature, duration, etc. in a cardiac, circulatory and/or respiratory support device. The control signal can in particular be a signal that automatically ends or starts the ablation or catheter ablation and/or the cardiac, circulatory and/or respiratory support, or controls or regulates the ablation temperature and/or the scope of support.

The bioimpedance is preferably continuously monitored during cardiac ablation or cardiac catheter ablation and/or cardiac, circulatory and/or respiratory support, the distance between two successive discrete bioimpedance measurements preferably being 5 seconds, particularly preferably 1 second. An averaging of, for example, 3 to 5 heart actions is also possible. A time-variable bioimpedance signal results from the individual bioimpedance measurements, e.g. in the form of an impedance cardiogram.

The bioimpedance of the first electrode measured at a specific time can be compared to the bioimpedance of the second electrode measured at that time. On the basis of the difference between the two values, a signal or information can be derived, which can influence or optimize the at least one parameter of the ablation or catheter ablation (such as energy output of heat or cold energy, laser energy, ultrasound energy, temperature, duration, spatial expansion, start, end, etc.) and/or cardiac, circulatory and/or respiratory support (such as volume, flow rate, oxygen and/or carbon dioxide transfer, temperature, duration, start, end, etc.). Excessive energy output, e.g. in the case of atrial fibrillation ablation, can lead to damage to the esophagus, which can lead to atrial esophageal fistulas between the left atrium and the esophagus. The aim of the optimization can e.g. be the detection and prevention of excessive energy output during ablation or catheter ablation.

It is also possible, on the basis of the individual bioimpedance measurements of the at least one first electrode and the at least one second electrode within a certain time interval, to determine characteristic signal parameters that can be compared with one another and on the basis of which a control signal can be generated. An exemplary characteristic signal parameter can be a weighted or unweighted mean value of the individual impedance measurements within a specific time interval. Other exemplary parameters can be the form of the time-variable impedance signals, the slope of certain signal sections, the distance between the signal maxima and/or signal minima, etc. In addition to the comparison of parameters in the time domain, parameters in the spectral domain (FFT, Spectro-Temporal Mapping, Wavelet Analysis, etc.) can be compared.

With several first electrodes, i.e. a plurality of electrodes arranged on the first side of the esophageal electrode probe, a first bioimpedance measurement signal can be formed from the individual measurement signals of all first electrodes, for example by forming a weighted or unweighted sum, a weighted or unweighted mean value, a median value, etc. The same applies to the case that there are several second electrodes: a second bioimpedance measurement signal can be formed from the individual measurement signals of all second electrodes.

According to a preferred example, the check signal (e.g. warning signal, status signal, control signal) is formed on the basis of the difference between the first bioimpedance measurement signal and the second bioimpedance measurement signal. The check signal can also be formed on the basis of the difference between electrocardiography signals, e.g. the difference between the electrocardiography signals of one or more near-heart and one or more remote from-heart esophageal electrodes, and from the combination of bioimpedance signals and electrocardiography signals. Furthermore, these transesophageal bioimpedance signals and/or electrocardiography signals can be formed with transthoracic bioimpedance signals and/or electrocardiography signals and/or intracardiac bioimpedance signals and/or electrocardiography signals.

The check signal may be a signal for terminating ablation or catheter ablation and/or cardiac, circulatory and/or respiratory support if the difference between the first bioimpedance measurement signal and the second bioimpedance measurement signal is equal to or greater than a predetermined threshold value. The threshold value can preferably be set individually by the examiner and can also depend on the type of catheter ablation and the experience of the rhythmologist. For example, the threshold can be greater than ten percent.

It is also possible, as an alternative or in addition, to compare temporal and/or spatial and/or spectral signal patterns and to generate a control signal based on the difference in the patterns.

The device can further comprise an ablation device for ablation or catheter ablation of at least part of the tissue surrounding the esophageal electrode probe, e.g. a cardiac ablation device or cardiac catheter ablation device. The ablation device can in particular be a catheter ablation device for catheter ablation of arrhythmias. The catheter ablation device can be adapted to carry out a high-frequency, a cryo-, an ultrasound or a laser ablation. The catheter ablation device can e.g. be a cardiac catheter or include a cardiac catheter. The catheter ablation device can be a device external to the probe or can be integrated in the probe itself. Furthermore, communication and/or a comparison of the signals between the esophageal electrode probe and intracardial ablation electrodes is conceivable.

The device can also comprise a cardiac, circulatory and/or respiratory support device for physiological cardiosynchronous, circulatory and/or respiratory support, which is in signal connection with the esophageal electrode probe. The cardiac, circulatory and/or respiratory support device can be used e.g. for the treatment of high-risk patients in interventional cardiology and for the treatment of cardiogenic shock.

The device can further comprise a display device that is in signal connection with the esophageal electrode probe and that is adapted to display the signals received from the at least part of the electrodes of the esophageal electrode probe, the result of an evaluation thereof and/or the check signal. The display device can also display signals that have been sent or are being sent to at least part of the electrodes of the esophageal electrode probe. For example, the display device can be in signal connection with the bioimpedance measuring device of the esophageal electrode probe and display the bioimpedance measurement signals and/or another signal derived therefrom (e.g. the difference thereof) in a suitable form.

It is also possible for several electrodes to be connected together by an electrical short circuit by means of an adapter or switching device to form one electrode for unipolar cardioversion or to form two electrodes for unipolar or bipolar cardioversion, the individual electrodes being made of conductive material, such as metal or conductive plastic/rubber.

Another aspect of the disclosure relates to a method for controlling or regulating a cardiac ablation device or cardiac catheter ablation device (ablation device for performing a cardiac ablation or cardiac catheter ablation) and/or a cardiac, circulatory and/or respiratory support device. The method comprises:
detecting a first bioimpedance measurement signal from at least one first electrode, the at least one first electrode being arranged on a side of an esophageal electrode probe facing the heart;
detecting a second bioimpedance measurement signal from at least one second electrode, the at least one second electrode being arranged on a side of the esophageal electrode probe facing away from the heart;
generating a control signal for controlling or regulating the cardiac ablation device or cardiac catheter ablation device and/or the cardiac, circulatory and/or respiratory support device on the basis of a comparison of the first bioimpedance measurement signal with the second bioimpedance measurement signal.

Alternatively or in addition, the method for controlling or regulating a cardiac ablation device or cardiac catheter ablation device (ablation device for performing a cardiac ablation or cardiac catheter ablation) and/or a cardiac, circulatory and/or respiratory support device can comprise the following steps:
detecting a first electrocardiography measurement signal from at least one first electrode, the at least one first electrode being arranged on a side of an esophageal electrode probe facing the heart; and
detecting a second electrocardiography measurement signal from at least one second electrode, the at least one second electrode being arranged on a side of the esophageal electrode probe facing away from the heart.

The method can further comprise:
generating a control signal for controlling or regulating the cardiac ablation device or cardiac catheter ablation device and/or the cardiac, circulatory and/or respiratory support device on the basis of a comparison of the first electrocardiography measurement signal with the second electrocardiography measurement signal.

The esophageal electrode probe can be an esophageal electrode probe according to one aspect of the disclosure. Accordingly, the method can comprise providing an esophageal electrode probe according to an aspect of the disclosure.

The control signal can in particular be the control signal described above. In particular, the control signal can be a signal for terminating an ablation or catheter ablation performed by the cardiac ablation device or cardiac catheter ablation device and/or cardiac, circulatory and/or respiratory support if the difference between the first bioimpedance measurement signal and the second bioimpedance measurement signal and/or the difference between the first electrocardiography measurement signal and the second electrocardiography measurement signal is equal to or greater than a predetermined threshold value.

Another aspect of the disclosure relates to a method for producing an esophageal electrode probe, e.g. an esophageal electrode probe according to an aspect of the disclosure. The method includes producing the esophageal electrode probe using a 3D printing method. The method can further comprise providing data for the 3D printing method. The data can include e.g. the shape, the dimensions, the electrodes and their arrangement, the materials for the individual components and/or other necessary data for the 3D printing process. The data can e.g. be stored in a database or on another suitable storage medium. The data can be in the form of 3D CAD data or in other suitable formats. The data can be created and/or tested using a heart model. The heart model can be created e.g. based on average or patient-specific patient and/or physiological data. An exemplary heart model will be described in detail below.

The disclosure will be explained below with reference to embodiments shown in the drawing. The drawings show:

### BRIEF DESCRIPTION OF THE DRAWINGS

FIG. 1 an exemplary device for transesophageal bioimpedance monitoring and/or for cardiac stimulation and/or ECG and/or cardiac neurostimulation, in particular for temporary transesophageal left-heart stimulation and/or left-heart electrocardiography and/or neurostimulation;

FIG. 2 an exemplary device for transesophageal bioimpedance monitoring in cardiac resynchronization therapy or for the optimization of a cardiac, circulatory and respiratory support device;

FIG. 3 an exemplary device for transesophageal cardioversion of atrial fibrillation and/or transesophageal left-heart electrocardiography;

FIG. 4 the results of a combination of temporary transesophageal high-frequency atrial stimulation and temporary transesophageal neurostimulation with reduction of the stimulus threshold of the transesophageal atrial stimulation;

FIG. 5 an exemplary esophageal electrode probe for temporary left-heart stimulation by means of bipolar transesophageal left-atrial and bipolar left-ventricular stimulation and temporary transesophageal neurostimulation;

FIG. 6 an exemplary esophageal electrode probe for temporary transesophageal left-ventricular stimulation with reduced electrode-myocardium distance and temporary transesophageal neurostimulation;

FIG. 7 an exemplary esophageal electrode probe for temporary transesophageal left-ventricular stimulation with reduced electrode-myocardium distance and temporary transesophageal neurostimulation with reduced electrode-spinal cord distance;

FIGS. 8 to 11 exemplary esophageal electrode probes with an inflatable catheter balloon for temporary transesophageal left-atrial and/or left-ventricular stimulation, and/or for electrocardiography, and/or for hemodynamic monitoring with reduced electrode-myocardium distance, and/or for temporary transesophageal neurostimulation with reduced electrode-spinal cord distance;

FIGS. 12 to 14 exemplary esophageal electrode probes for temporary transesophageal stimulation, and/or electrocardiography, and/or bioimpedance measurement, and/or catheter ablation and/or cardioversion and/or cardiac stimulation and/or cardiac neurostimulation and/or temporary transesophageal neurostimulation without catheter balloon (FIG. 12), with an uninflated catheter balloon (FIG. 13) and with an inflated catheter balloon (FIG. 14);

FIG. 15 an exemplary esophageal electrode probe for neurostimulation and/or bipolar DC AF termination;

FIG. 16 an exemplary esophageal electrode probe for neurostimulation and/or unipolar DC AF termination;

FIG. 17 an exemplary 3D CAD heart model with an esophageal electrode probe;

FIG. 18 an exemplary cardiac neurostimulation in left-atrial and left-ventricular stimulation; and

FIG. 19 an exemplary cardiac neurostimulation and transthoracic and transesophageal electrocardiography at sinus rhythm and bundle branch block.

### DETAILED DESCRIPTION

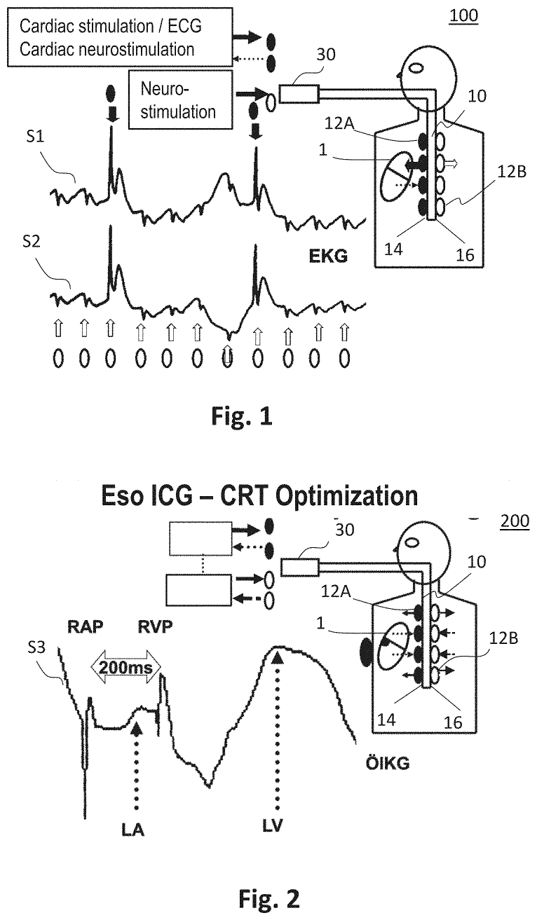

FIG. 1 schematically shows an exemplary device for transesophageal bioimpedance monitoring and/or for further measurements and treatments, such as for cardiac stimulation, ECG and/or cardiac neurostimulation, in particular for temporary transesophageal left-heart stimulation and/or left-cardiac electrocardiography and/or neurostimulation 100 (as an example of a device for transesophageal cardiological treatment and/or diagnosis). The device 100 includes an esophageal electrode probe 10 with a plurality of electrodes 12A on the side 14 facing the heart 1 (near-heart) and with a plurality of electrodes 12B on the side 16 of the esophagus electrode probe 10 facing away from the heart. In the figures, the electrodes 12A on the near-heart side 14 of the esophageal electrode probe 10 are represented by black filled ellipses or circles and the electrodes 12B of the esophageal electrode probe 10 on the side 16 facing away from the heart are represented by unfilled ellipses or circles. The near-heart side and the side of the esophageal electrode probe 10 facing away from the heart can e.g. be marked by appropriate markings on the esophageal electrode probe 10, which enable a controlled placement of the probe in relation to the heart 1.

The electrodes 12A and 12B are each arranged in rows in the longitudinal direction or along the length of the esophageal electrode probe 10, with at least one row of electrodes 12A being arranged on the near-heart side 14 and at least one row of electrodes 12B being arranged on the side 16 of the esophageal electrode probe 10 facing away from the heart. The electrodes 12 comprise electrodes for bioimpedance measurement, which are arranged both on the near-heart side 14 and on the side 16 of the esophagus electrode probe facing away from the heart, electrodes for temporary transesophageal left-heart stimulation and/or left-heart cardiography and electrodes for neurostimulation, which are on the side 16 of the esophagus electrode probe 10 facing away from the heart. The neurostimulation can be used in particular for pain reduction in the case of transesophageal electrical stimulation and/or for the reduction of the stimulus threshold in the case of transesophageal left-ventricular and left-atrial stimulation, and can be carried out e.g. by high-frequency electrical signals with a frequency of 100 bpm to 1200 bpm, preferably from 100 bpm to 300 bpm, a strength of about 5V to 50V with a pulse width of 2 to 20 milliseconds for a duration of 2 seconds to 30 seconds.

The esophageal electrode probe 10 can also include additional electrodes or sensors, such as electrodes for transesophageal left-ventricular chamber stimulation and/or transesophageal left-atrial atrial stimulation on the near-heart side 14 of the esophageal electrode probe 10 or electrocardiography electrodes (ECG electrodes) on the near-heart side 14 of the esophageal electrode probe 10 for left-cardiac electrocardiography.

The individual electrodes 12 can be conventional electrodes that are at least partially made of a conductive material. For example, the electrodes 12 can have a substantially semi-spherical or semi-cylindrical shape, with the curved surface coming into contact with the patient's esophagus.

The electrodes 12 are connected to a control and evaluation device 30 via signal lines. The control and evaluation device 30 can be an external device or a device integrated in the esophageal electrode probe 10. In the device shown in FIG. 1, the control and evaluation device 30 is arranged outside the esophageal electrode probe 10.

Two or more of the electrodes 12 can be connected together. For example, two interconnected and/or controlled electrodes 12 can be used as a bipolar atrial electrode for transesophageal atrial stimulation or perception or as a bipolar ventricular electrode for transesophageal ventricular stimulation or perception. Four electrodes 12 can be connected together to form a unipolar electrode for unipolar cardioversion of atrial flutter or atrial fibrillation with a transthoracic or intracardial counterelectrode. By parallel operation of two distal and two proximal electrodes, for example, a transesophageal bipolar cardioversion is possible.

The electrodes 12 are in signal connection with a control and/or evaluation device 30, which evaluates the signals from the electrodes 12 (e.g. from the electrodes for bioimpedance measurement) and/or sends signals (e.g. control signals) to the electrodes 12 and possibly further devices. The signals received by the electrodes and/or the result of the evaluation thereof can be displayed on a display device. The control and/or evaluation device 30 is also adapted to generate evaluation signals (such as the warning signals, status signals and/or control signals described above) on the basis of the received signals in order to influence or control the progress of a catheter ablation, a cardiostimulation, a cardiac, circulatory and/or respiratory support device, and/or a neurostimulation. FIG. 1 further shows two electrocardiography signals S1 and S2 with two high-frequency electrostimulations.

FIG. 2 schematically shows an exemplary device 200 for transesophageal bioimpedance monitoring in cardiac resynchronization therapy or for optimization of a cardiac, circulatory and respiratory support device, in particular in high-frequency, cryo-, ultrasound or laser ablation of atrial fibrillation by isolation of the respiratory veins in the left atrium. The device 200 is an example of a device for transesophageal cardiological treatment and/or diagnosis.

The device 200 includes an esophageal electrode probe 10 with a plurality of electrodes 12 arranged in rows for bioimpedance measurement, a first row of electrodes 12A being arranged on the near-heart side 14 and a row of electrodes 12B being arranged on the side 16 of the esophagus electrode probe 10 facing away from the heart. The electrodes 12 are in signal connection with the control and evaluation device 30.

The device for transesophageal bioimpedance monitoring 200 and in particular the control and evaluation device 30 are adapted to continuously monitor tissue changes with the electrodes 12 facing the heart, to compare the bioimpedance measurement of the electrodes 12A facing the heart with the bioimpedance measurement of the electrodes 12B facing away from the heart and to derive and/or display information therefrom. At the same time, transesophageal hemodynamic monitoring can be performed.

FIG. 2 shows an exemplary transesophageal impedance cardiography signal S3 of a biventricular stimulation. For example, this can be used in respiratory vein isolation to treat atrial fibrillation in an implanted cardiac resynchronization therapy (CRT) system. In particular, FIG. 2 shows the right-atrial (RAP) and right-ventricular (RVP) stimulation pulse with a 200 ms atrioventricular delay of the implanted CRT system and the left-atrial (LA) and left-ventricular ECG signals with optimal biventricular stimulation with LA signal temporally before the RVP signal.

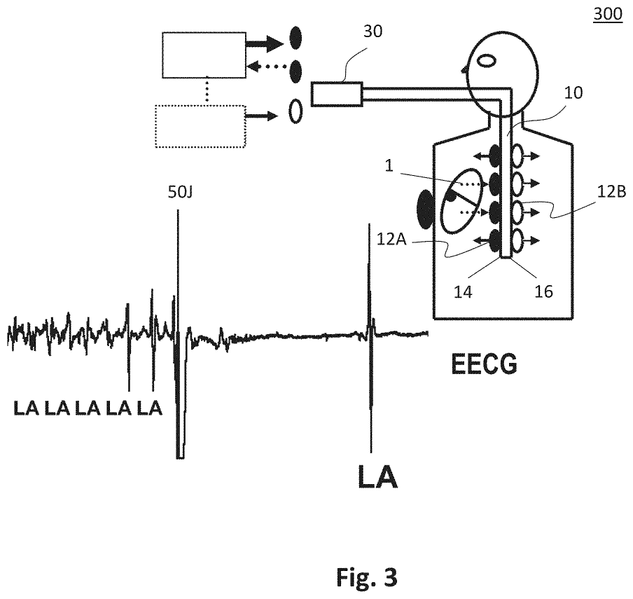

FIG. 3 schematically shows an exemplary device for transesophageal neurostimulation and cardiostimulation 300, in particular for transesophageal left-atrial and/or left-ventricular stimulation and electrocardiography as part of the diagnosis and therapy of bradycardic and tachycardiac arrhythmias, e.g. unitiation and termination of AV node reentry tachycardia (AVNRT), AV reentry tachycardia (AVRT) and atrial flutter, or for transesophageal left-ventricular stimulation as part of a temporary cardiac resynchronization therapy. The device 300 is an example of a device for transesophageal cardiological treatment and/or diagnosis.

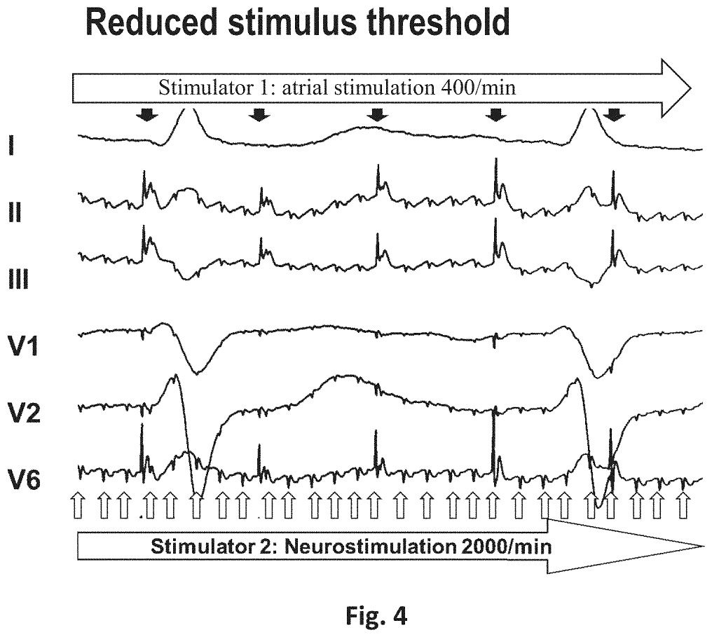

The device 300 includes an esophageal electrode probe 10 with a plurality of electrodes 12 for neurostimulation on the side 16 of the esophageal electrode probe 10 facing away from the heart 1 and with a plurality of electrodes on the side 14 of the esophageal electrode probe 10 facing the heart for transesophageal electrical cardioversion by DC energy output, for example of 50J, for atrial flutter and atrial fibrillation. The esophageal electrode probe 10 may also comprise additional electrodes, such as ECG electrodes arranged on the side 14 of the esophageal electrode probe 10 facing the heart 1. The electrodes 12 are in signal connection with the control and evaluation device 30. Electrical signals with a frequency of 100 bpm to 3000 bpm, preferably from 1500 bpm to 2000 bpm and a strength of 5V to 100V are applied to the electrodes for neurostimulation for a duration of 3 to 30 seconds. The transesophageal neurostimulation enables pain reduction of the transesophageal cardiac stimulation, as shown in FIG. 4. In particular, FIG. 4a shows transthoracic ECG leads I, II, III, V1, V2 and V6 for high-frequency left-atrial stimulation with 400 bpm with stimulator 1 and high-frequency non-cardiac stimulation with 2000 bpm with stimulator 2.

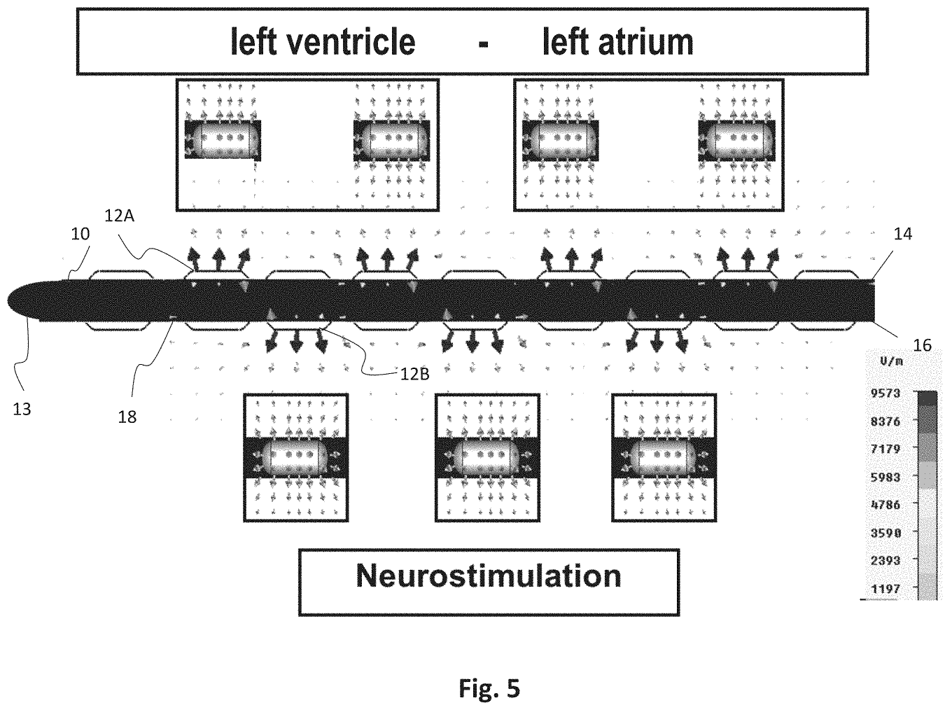

FIG. 5 shows an exemplary esophageal electrode probe 10 for temporary transesophageal bipolar left-atrial and left-ventricular stimulation and noncardiac neurostimulation. The esophageal electrode probe 10 includes an elongated, essentially cylindrical, flexible probe body 18 with a distal end 13 and a proximal end. The longitudinal axis of the probe body 18 substantially coincides with the insertion direction of the probe. A plurality of electrodes 12 are arranged in rows on the probe body 18 between the distal and proximal ends. Each row of electrodes extends substantially in the longitudinal direction of the probe 10. The electrodes 12B on the side of the esophageal electrode probe 10 facing away from the heart comprise electrodes for neurostimulation. The electrodes 12A on the side of the esophageal electrode probe 10 facing the heart comprise electrodes 12A for bipolar left-atrial and bipolar left-ventricular stimulation.

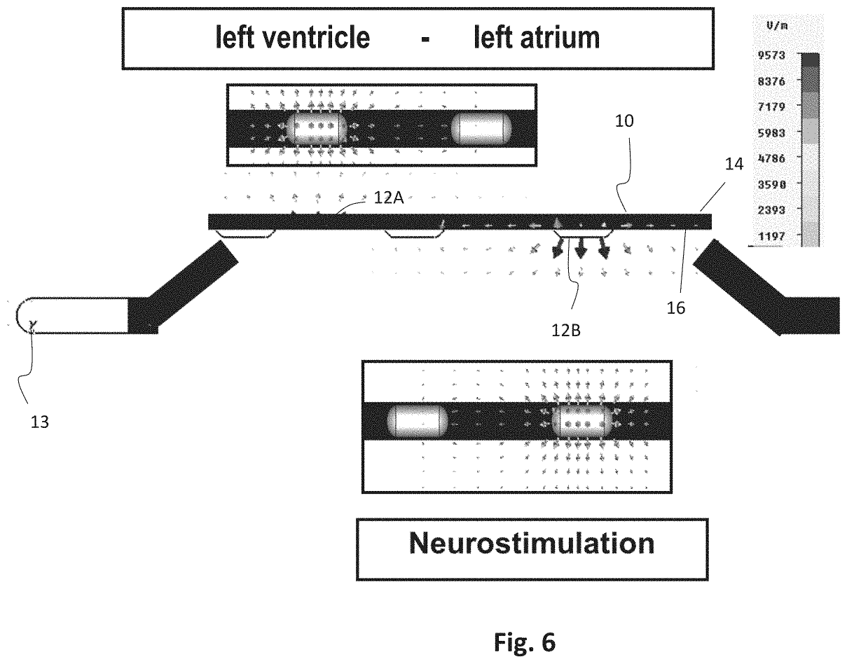

FIG. 6 shows another exemplary esophageal electrode probe 10 with flat electrodes 12A for left-heart stimulation and electrodes 12B for neurostimulation with a large electrode-spinal cord distance on the side of the esophageal electrode probe 10 facing away from the heart and a device for changing the electrode-myocardium distance, e.g. with a correspondingly pre-bent mandrin/stylet or with shape memory material.

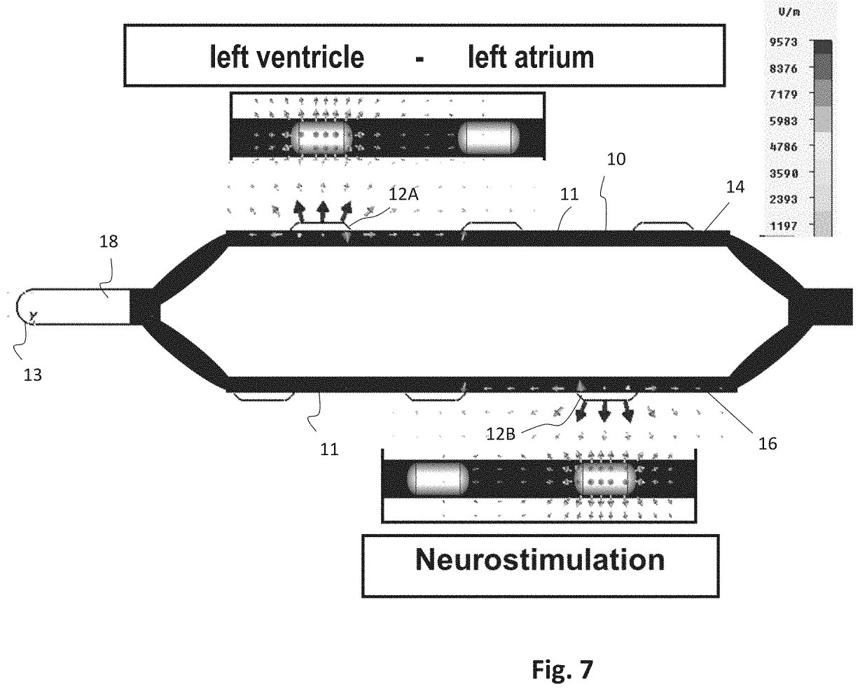

FIG. 7 shows an exemplary esophageal electrode probe 10 for left-heart stimulation and neurostimulation with a plurality of elongated segments 11 with electrodes 12, which can reduce the electrode-myocardium distance. The elongated segments 11 are arranged similar to the segments of an umbrella that can be opened. The electrodes 12B on the side facing away from the heart comprise electrodes for neurostimulation and bioimpedance measurement. The electrodes on the side facing the heart include comprise electrodes for left-heart stimulation, ECG and bioimpedance measurement. The arrangement of the electrodes 12 is similar to the esophageal electrode probe 10 shown in FIG. 5. Also, the esophageal electrode probe 10 includes a device for changing the electrode-myocardium distance and the electrode-spinal cord distance, e.g. with a pre-bent mandrin/stylet or with shape memory material.

The electrodes 12 have a substantially semi-spherical or semi-cylindrical shape with a substantially plane surface and a conductive curved surface. In particular, electrodes 12B for neurosimulation and electrodes 12A for cardiostimulation are attached on the side 16 facing away from the heart and on the side facing the heart, respectively.

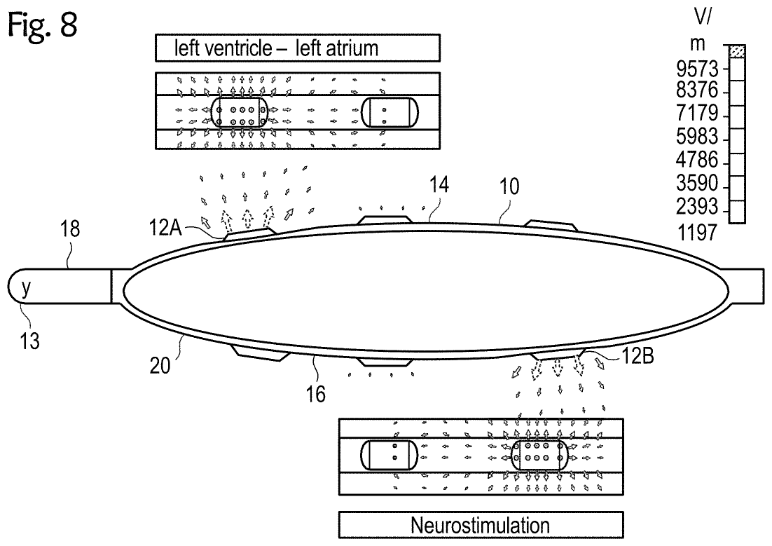

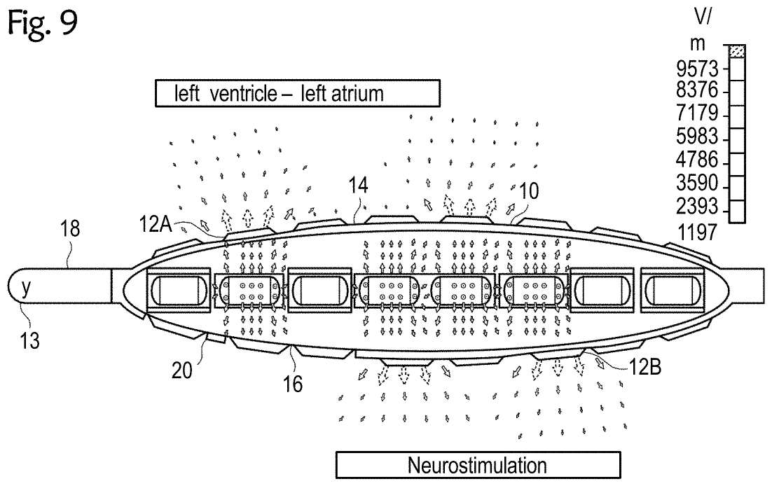

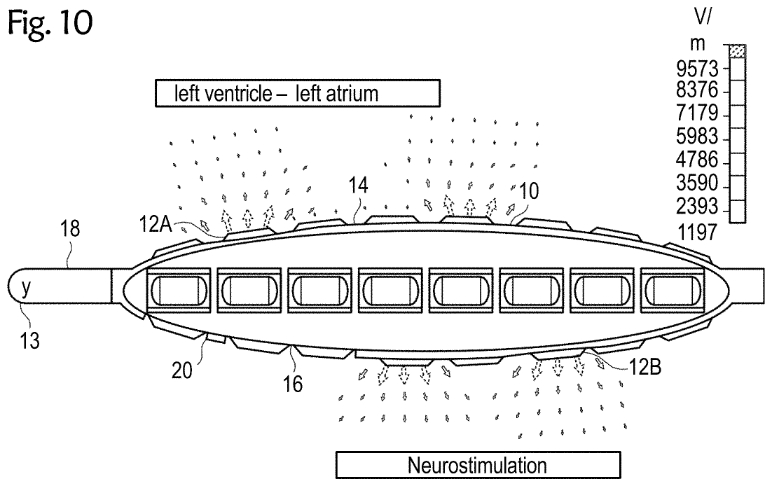

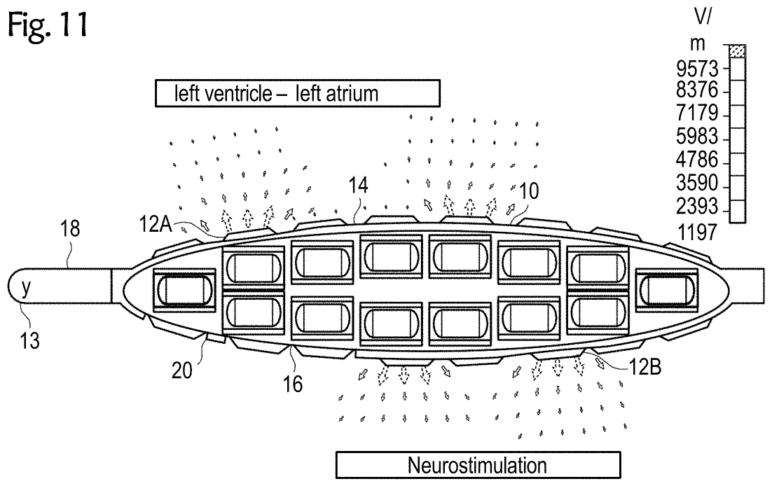

FIGS. 8 to 11 each show other exemplary esophageal electrode probes 10 for temporary transesophageal left-atrial and/or left-ventricular stimulation, and/or for electrocardiography, and/or for hemodynamic monitoring with a reduced electrode-myocardium distance and/or for temporary transesophageal neurostimulation with reduced electrode/spinal cord distance. The esophageal electrode probes 10 have an inflatable catheter balloon 20 on which electrodes 12 are attached.

The catheter balloon 20, which is formed from a biocompatible elastic material, is attached to the cylindrical probe body 18. For example, the catheter balloon 10 can be attached to the distal and proximal ends of the probe body 18. When the esophageal electrode probe 10 is in its correct position in the patient's esophagus, the catheter balloon 20 is inflated so that it comes into close contact with the patient's esophagus. FIGS. 8 to 11 each show the esophageal electrode probe 10 with the inflated catheter balloon 20. In this state, the conductive electrode surface of the electrodes 12 comes into low-resistance contact with the patient's esophagus, so that the stimulations and/or measurements can be carried out.

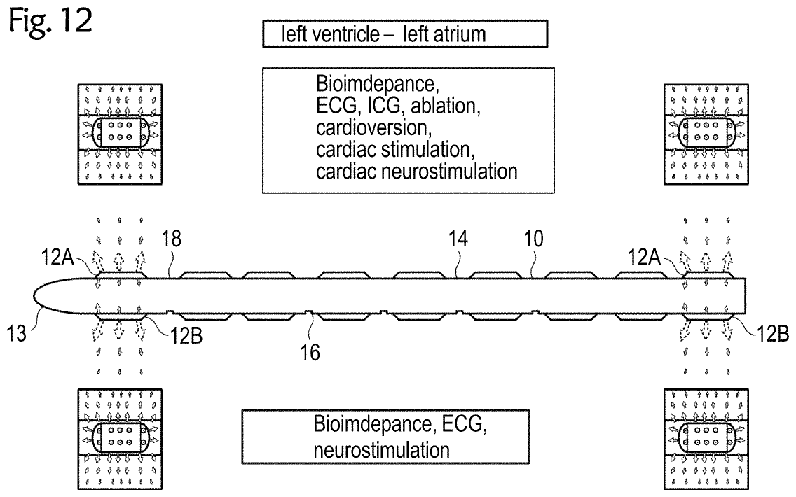

FIG. 12 shows an exemplary esophageal electrode probe 10 for bioimpedance measurement and optionally for further measurements and/or treatments, such as for ECG, ICG, catheter ablation, cardioversion, cardiac stimulation, cardiac neurostimulation, in particular for directional left-heart stimulation without the possibility of reducing the electrode-myocardium distance. The neurostimulation can optionally be carried out as cardiac neurostimulation in the direction of the heart and/or non-cardiac neurostimulation in the direction of the spine.

The esophageal electrode probe 10 has a plurality of electrodes 12 arranged in rows in the longitudinal direction of the probe body 18. The electrodes 12A on the side 14 of the esophageal electrode probe 10 facing the heart comprise electrodes for bioimpedance measurement and optionally electrodes for ICG, ECG, cardioversion, catheter ablation, neurostimulation and/or cardiac stimulation. The electrodes 12B on the side 16 of the esophageal electrode probe 10 facing away from the heart comprise electrodes for bioimpedance measurement and optionally also electrodes for ECG and neurostimulation. The proximal electrodes 12A are electrodes for unipolar or bipolar left-ventricular stimulation and electrocardiography and bioimpedance, and the proximal electrodes 12A are electrodes for unipolar or bipolar left-atrial stimulation and electrocardiography and bioimpedance without a catheter balloon.

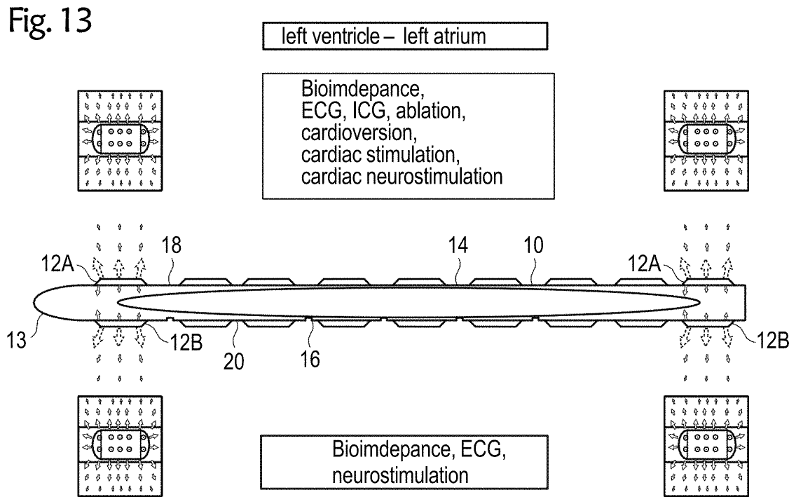

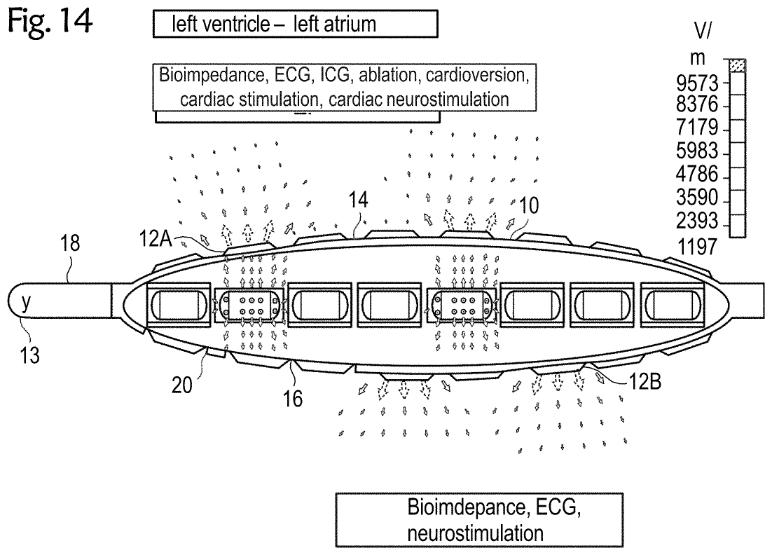

FIGS. 13 and 14 each show other exemplary esophageal electrode probes 10 for bioimpedance measurement and optionally for further measurements and/or treatments, such as for left-heart stimulation, left-heart electrocardiography, left-heart bioimpedance, and/or neurostimulation. The neurostimulation can optionally be carried out as cardiac neurostimulation in the direction of the heart and/or non-cardiac neurostimulation in the direction of the spine.

FIG. 13 shows an exemplary esophageal electrode probe 10 with an uninflated catheter balloon 20. FIG. 14 shows an exemplary esophageal electrode probe with an inflated catheter balloon 20.

The esophageal electrode probe 10 has four symmetrically arranged rows of electrodes for stimulation, ECG, bioimpedance, cardiac neurostimulation, catheter ablation, etc. The difference to the previous probes is that the bipolar stimulation and/or electrocardiography/impedance between two neighboring electrodes can be realized in neighboring electrode rows. This allows, for example, more local ECGs to be detected and the left heart to be stimulated more locally.

Two or more of the electrodes 12 can be switched together and/or controlled as described above.

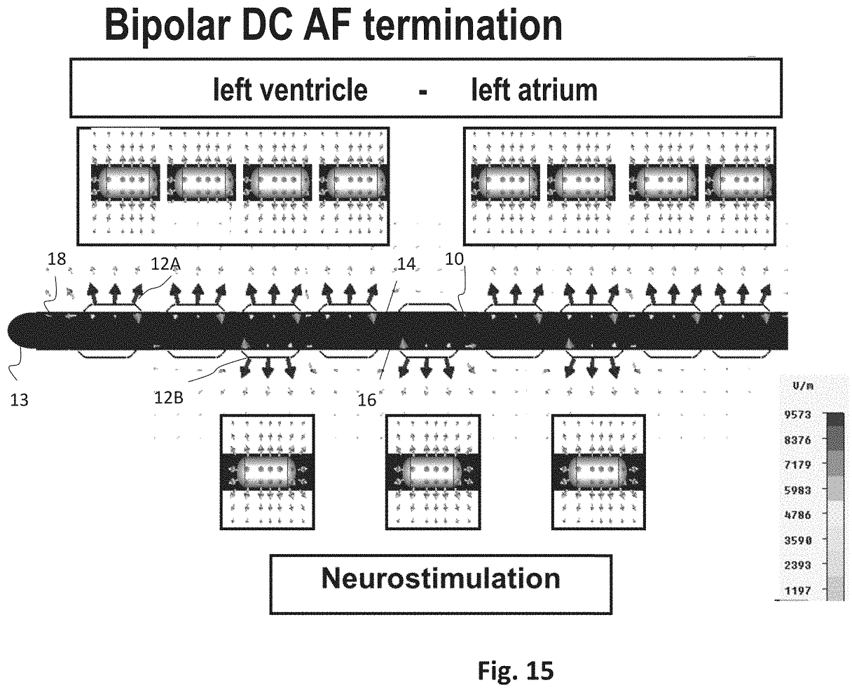

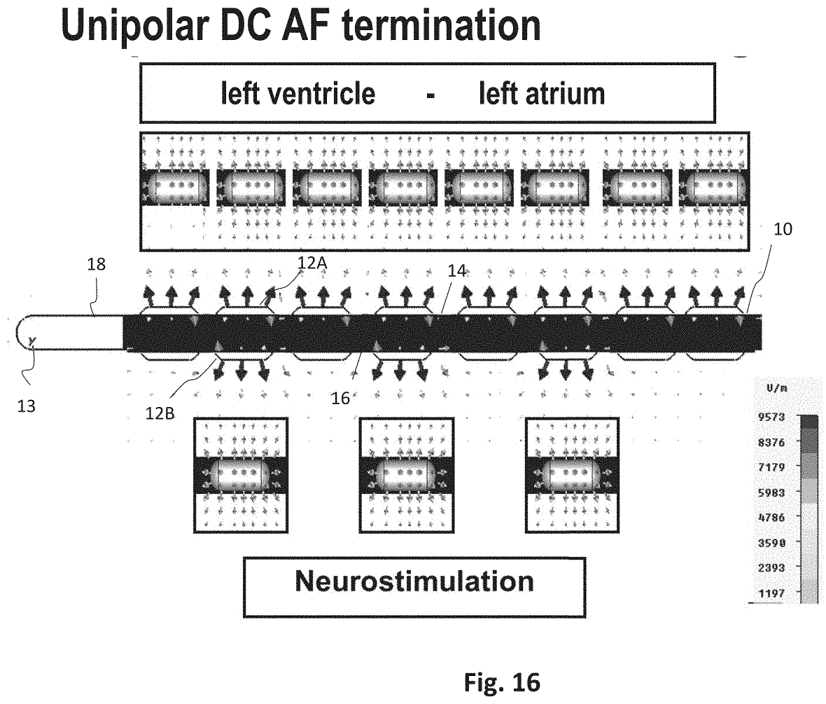

FIG. 15 shows an exemplary esophageal electrode probe 10 for bipolar DC cardioversion for the termination of atrial fibrillation, atrial flutter and combination with stimulation, ECG and impedance. FIG. 16 shows an exemplary esophageal electrode probe 10 for unipolar DC cardioversion for the termination of atrial fibrillation, atrial flutter and combination with stimulation, ECG and impedance.

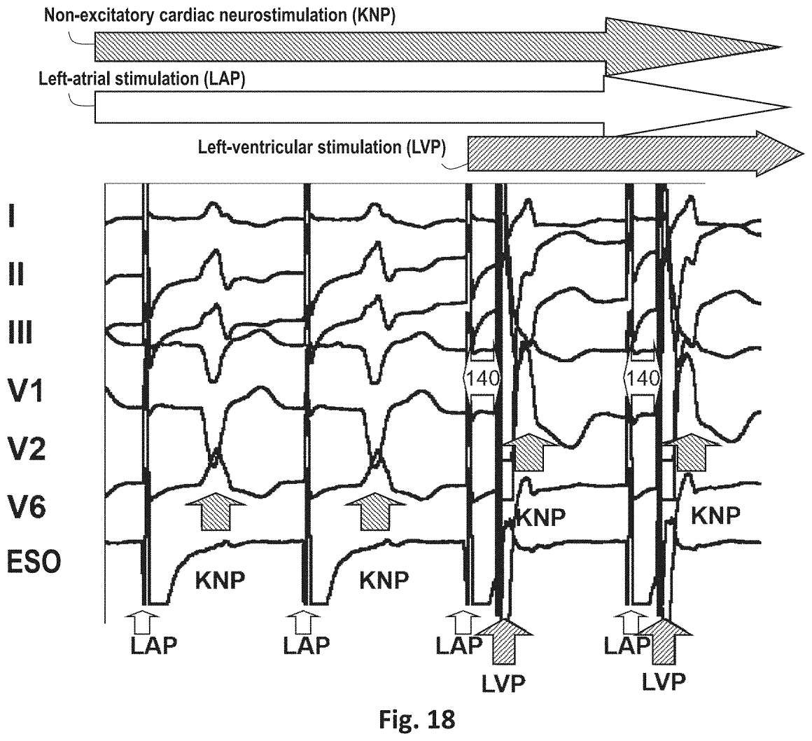

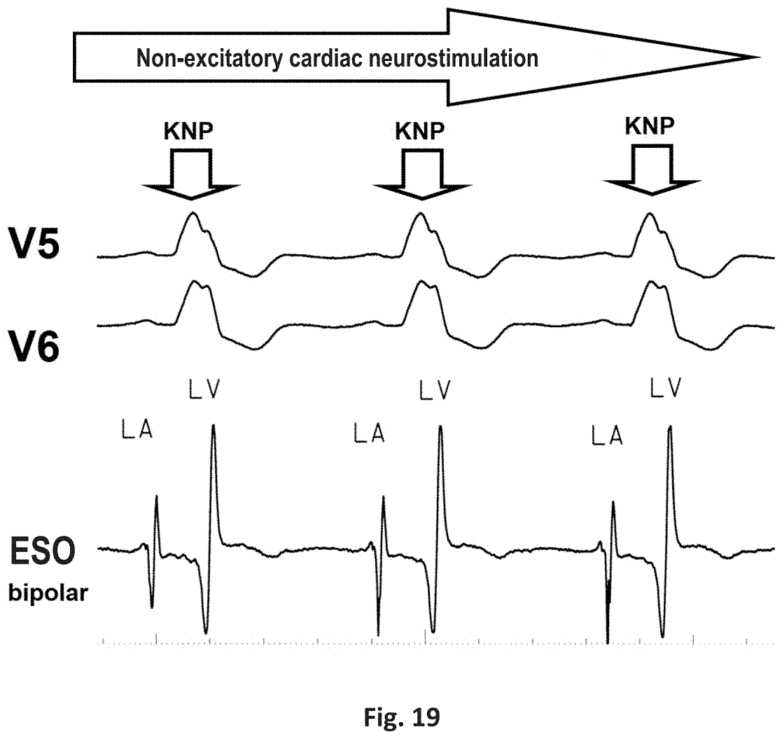

FIGS. 18 and 19 each show examples of cardiac neurostimulations that can be carried out using the esophageal electrode probes 10 described above. In particular, FIGS. 18 and 19 show the transthoracic ECGs with the leads I, II, III, V1, V2, V5 and V6. FIG. 18 shows an exemplary cardiac neurostimulation with left-atrial and left-ventricular stimulation. FIG. 19 shows an exemplary cardiac neurostimulation and transthoracic and transesophageal electrocardiography at sinus rhythm and bundle branch block. The cardiac neurostimulation can be a non-excitatory cardiac neurostimulation (KNP), i.e. a high-frequency directional stimulation in the absolute ventricular refractory period, the stimulation being delivered within the QRS complex. The signals for control and regulation realize the pulse output in the QRS complex and prevent a pulse output outsides the QRS complex.

The cardiological treatments and/or measurements with the esophageal electrode probes 10 according to different aspects of the disclosure can be simulated using a digital heart model, e.g. based on 3D CAD technology. An anatomically correct 3D CAD heart rhythm model (HRM) for the simulation of electrophysiological examinations (EPU) and radio frequency (HF) ablations will be described below. This model can be used to electrically and thermally simulate complex cardiac rhythmological structures, intracardiac and esophageal electrode catheters and cardiac pacemaker electrodes. This is of great importance for the individualized optimization of the catheters and the catheter ablation process and of cardiac rhythm implants and for the optimization of lengthy and costly clinical studies. Likewise, the risk of endangering patients is reduced to a minimum and can be used in the context of teaching and research in the field of diagnosis and therapy of arrhythmias. With the help of the proposed 3D heart rhythm model, esophageal electrode probes can be produced in a patient-optimized and individualized way using the 3D printing technology as a prototype or series product.

The proposed 3D heart rhythm model includes myocardium, cardiac clamps, excitation formation, stimulus conduction, esophagus and intracardiac electrode catheter (as an example of an esophageal electrode probe) for the simulation of electrophysiological examinations (EPU), high-frequency (HF) ablation, cardiac pacemaker therapy and various bradycardic and tachycardic arrhythmias. In particular, in the 3D heart rhythm model, sinus nodes, Bachmann bundles, AV nodes, His bundles and right and left-ventricular Tawara branches are modeled. For example, the anatomy can be modeled to scale on the basis of MRT images and anatomical sectional images. Various electrode catheters and in particular esophageal electrode probes can also be modeled and positioned at suitable locations in the heart model. The materials used for the cardiac catheter and/or the tissue parameters of the heart anatomy and rhythmology can be read out from a database, wherein the database may be part of the simulation software.

The proposed heart rhythm model can be based for example on CST STUDIO SUITE®, a simulation software from CST Computer Simulation Technology AG, Darmstadt, with which a variety of electromagnetic simulations can be carried out. Another advantage is that a large number of different material parameters are available. For example, the Material Library from CST contains a variety of materials related to human body tissue, wherein in these materials the necessary parameters such as electrical conductivity or heat capacity are contained. Of course, other simulation software can also be used.

To realize the heart model, the four ventricles and the heart's stimulus conduction and excitation formation system are modeled using material parameters (such as electrical conductivity, heat capacity, etc.) that relate to the human body tissue. Tissue cooling is preferably taken into account in the heart model by a calculated blood flow and metabolism. Furthermore, changes in the impedance of the tissue can also be taken into account.

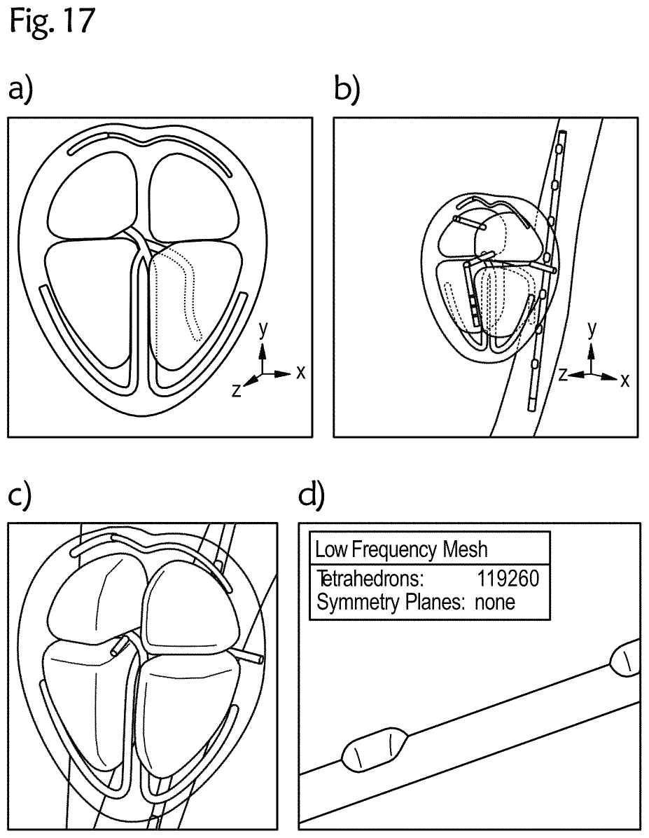

FIG. 17 shows an exemplary 3D CAD heart model, which uses a tetrahedral mesh, wherein FIG. 17a shows the 3D CAD heart rhythm model with excitation conduction, FIG. 17b the heart model with cardiac catheters positioned, FIG. 17c the tetrahedral mesh of the ventricles and the excitation conduction, and FIG. 17d a section of the tetrahedral mesh of the esophageal catheter or the esophageal electrode probe.

In particular, the heart model has the following features:
3D modeling of the organs using a spline function, e.g. a tetrahedral mesh;
complete cardiac lead (sinus node, AV node, Tawara branch, Bachmann bundle) by spline functions;
positioning of the cardiac lead based on averaged MRT data;
realization of the electrical conductivity through defined voltage paths along the cardiac lead;
modeling of the esophagus by spline functions using MRT data;
modeling of different catheters, such as multipolar electrode catheters;
modeling of different bradycardia and tachycardia arrhythmias;
modeling of electrostimulation, neurostimulation and/or electrocardiography;
modeling of different ablations or catheter ablations, e.g. HF ablations;
creation of a sine node signal based on EPU data by superimposing a defined trapezoidal signal and an action potential;
definition of electrical heart excitation along the cardiac lead;
modeling a realistic heart action by determining the temporal sequence of the signals and their amplitudes;
definition of monitoring parameters with tested mesh parameters and resolution properties of the monitoring functions;
deriving of the signals by defined 1D “monitors” on the catheters;
implementation of ablation therapy by superimposing a high-frequency signal and an energy;
interfaces and data formats for 3D printing of patient-specific heart rhythm models with and without cardiac catheter and/or esophageal electrode probe and/or electromagnetic and/or thermal field profiles for medical care, teaching and research;
interfaces and data formats for 3D printing of esophageal electrode probes as prototypes and/or after approval of 3D print as series products.

The heart model in particular enables temporal simulations in the low frequency range. Due to the possibility to apply electrical potentials independent of the material and to define voltage paths, the heart model is ideally suited for the simulation of excitation conductions within the heart and for the simulation of electrical heart stimulation and electrocardiography with intracardial and transesophageal electrode catheters.

The heart model also enables the electrical or other properties to be monitored at defined points. The function of monitoring at defined points enables the derivation of simulated eigen-signals of the heart with the help of different electrodes of a multipolar electrode catheter. The temporal representation of an electrical cardiac activity can be visualized as an E-field using the LF Time Domain Solver.

Furthermore, different excitation signals can be created within the heart model, which enables the reconstruction of different heart rhythms. A thermal simulation can also be carried out, wherein heat and power sources are simulated and, depending on the desired result, are calculated statically or in the time domain over a defined period of time. By simulation of power sources in the time domain, it was possible to present a therapy in the form of HF ablation by the possibility of defining a high-frequency sinusoidal signal. With 3D printing, heart rhythm models and electrode models it is possible to create patient-specific heart rhythm models with and without cardiac catheters and/or esophageal electrode probes and/or electromagnetic and/or thermal field profiles for medical care, teaching and research.

### REFERENCE NUMERAL LIST

1 heart
10 esophageal electrode probe
11 elongated segments
12 electrodes
12A electrodes on the side of the esophageal electrode probe facing the heart
12B electrodes on the side of the esophageal electrode probe facing away from the heart
13 distal end of the esophageal electrode probe
14 side of the esophageal electrode probe facing the heart
16 side of the esophageal electrode probe facing away from the heart
18 probe body
20 catheter balloon
30 control and/or evaluation device
100 device for transesophageal bioimpedance monitoring and for neurostimulation
200 device for transesophageal bioimpedance monitoring
300 device for transesophageal neurostimulation and cardiostimulation;
S1 to S3 signals

1. An esophageal electrode probe comprising:
a bioimpedance measuring device for measuring the bioimpedance of at least part of tissue surrounding the esophageal electrode probe, wherein the bioimpedance measuring device comprises at least one first electrode and at least one second electrode, wherein the at least one first electrode is arranged on a side of the esophageal electrode probe facing the heart, and wherein the at least one second electrode is arranged on a side of the esophageal electrode probe facing away from the heart.

2. The esophageal electrode probe according to claim 1, further comprising a neurostimulation device, wherein the neurostimulation device comprises at least one electrode for transesophageal neurostimulation of the at least part of the tissue surrounding the esophageal electrode probe by electric pulses with a frequency of 100 bpm to 3000 bpm and a strength of 5V to 100V and a duration of 3 seconds to 10 minutes, wherein the at least one electrode for neurostimulation is arranged on the side (16) of the esophageal electrode probe facing away from the heart.

3. The esophageal electrode probe according to claim 1, further comprising
a cylindrical probe body and an inflatable catheter balloon attached to the probe body, wherein the electrodes of the bioimpedance measuring device and/or the neurostimulation device are arranged on the catheter balloon.

4. The esophageal electrode probe according to claim 1, further comprising at least one of:
a stimulation device with at least one electrode for transesophageal cardiac stimulation;
an electrography device with at least one electrode for electrography measurement;
an echocardiography device with at least one ultrasound sensor for echocardiography measurement;
a temperature measuring device with at least one temperature sensor; or
a pH value measuring device with at least one pH value sensor for pH value measurement.

5. The esophageal electrode probe according to claim 1, wherein the esophageal electrode probe comprises an electrography device with at least one first and at least one second electrode for electrography measurement, wherein the at least one first electrode for electrography measurement is arranged on a side of the esophageal electrode probe facing the heart, and the at least one second electrode for electrography measurement is arranged on a side of the esophageal electrode probe facing away from the heart.

6. A device for transesophageal cardiological treatment and/or for transesophageal cardiological diagnosis, comprising:
an esophageal electrode probe that includes a bioimpedance measuring device for measuring the bioimpedance of at least part of tissue surrounding the esophageal electrode probe, wherein the bioimpedance measuring device comprises at least one first electrode and at least one second electrode, wherein the at least one first electrode is arranged on a side of the esophageal electrode probe facing the heart, and wherein the at least one second electrode is arranged on a side of the esophageal electrode probe facing away from the heart;
a control and/or evaluation device in signal connection with the bioimpedance measuring device, wherein the control and/or evaluation device is adapted to receive and compare a first bioimpedance measurement signal from the at least one first electrode and a second bioimpedance measurement signal from the at least one second electrode, and to generate a check signal on the basis of the comparison.

7. The device according to claim 6, wherein the esophageal electrode probe includes an electrography device with at least one first and at least one second electrode for electrography measurement, wherein the at least one first electrode for electrography measurement is arranged on a side of the esophageal electrode probe facing the heart, and the at least one second electrode for electrography measurement is arranged on a side of the esophageal electrode probe facing away from the heart, and wherein
the control and/or evaluation device is adapted to receive and compare a first electrocardiography measurement signal from the at least one first electrode and a second electrocardiography measurement signal from the at least one second electrode of the electrography device, and to generate the check signal on the basis of the comparison.

8. The device according to claim 6, further comprising at least one of:
an ablation device for performing a cardiac catheter ablation, wherein the ablation device is in signal connection with the control and/or evaluation device; or
a cardiac, circulatory and/or respiratory support device for cardiosynchronous cardiac, circulatory and/or respiratory support, wherein the cardiac, circulatory and/or respiratory support device is in signal connection with the control and/or evaluation device.

9. The device according to claim 8, wherein the check signal:
is a status signal indicating the status or value of at least one parameter of the cardiac catheter ablation and/or cardiac, circulatory and/or respiratory support; and/or
is a warning signal indicating that at least one parameter of a cardiac catheter ablation and/or cardiac, circulatory and/or respiratory support is outside an admissible value range or is greater or smaller than a predetermined threshold value; and/or
is a control signal for controlling or regulating an ablation device and/or a cardiac, circulatory and/or respiratory support device.

10. The device according to claim 6, wherein the check signal is a signal for terminating the cardiac catheter ablation and/or the cardiac, circulatory and/or respiratory support, and wherein the check signal is generated when the difference between the first bioimpedance measurement signal and the second bioimpedance measurement signal is equal to or greater than a predetermined threshold value.

11. The device according to claim 6, further comprising a display device in signal connection with the esophageal electrode probe, wherein the display device is adapted to display the first bioimpedance measurement signal and/or the second bioimpedance measurement signal and/or the check signal.

12. A method for controlling or regulating an ablation device for performing a cardiac catheter ablation and/or a cardiac, circulatory and/or respiratory support device for cardiosynchronous cardiac, circulatory and/or respiratory support, the method comprising:
detecting a first bioimpedance measurement signal from at least one first electrode, wherein the at least one first electrode is arranged on a side of an esophageal electrode probe facing a heart;
detecting a second bioimpedance measurement signal from at least one second electrode, wherein the at least one second electrode is arranged on a side of the esophageal electrode probe facing away from the heart;
generating a control signal for controlling or regulating the ablation device and/or the cardiac, circulatory and/or lung support device on the basis of a comparison of the first bioimpedance measurement signal with the second bioimpedance measurement signal.

13. The method of claim 12, further comprising:
detecting a first electrocardiography measurement signal from at least one first electrode, wherein the at least one first electrode is arranged on the side of an esophageal electrode probe facing the heart; and
detecting a second electrocardiography measurement signal from at least one second electrode, wherein the at least one second electrode is arranged on the side of the esophageal electrode probe facing away from the heart; wherein
the control signal is further generated on the basis of a comparison of the first electrocardiography measurement signal with the second electrocardiography measurement signal.

14. The method according to claim 12, wherein the control signal is a signal for terminating a catheter ablation performed by the ablation device or a cardiac, circulatory and/or respiratory support, and wherein the control signal is generated when the difference between the first bioimpedance measurement signal and the second bioimpedance measurement signal is equal to or greater than a predetermined threshold value.

15. The method of claim 12, wherein the esophageal electrode probe is produced by a 3D printing method, and wherein the esophageal electrode probe includes a bioimpedance measuring device for measuring the bioimpedance of at least part of tissue surrounding the esophageal electrode probe, wherein the bioimpedance measuring device comprises at least one first electrode and at least one second electrode, wherein the at least one first electrode is arranged on a side of the esophageal electrode probe facing the heart, and wherein the at least one second electrode is arranged on a side of the esophageal electrode probe facing away from the heart.
